(19) United States
(12) Patent Application Publication
(10) Pub. No.: US 2020/0046860 A1
(43) Pub. Date: Feb. 13, 2020

(54) Systems and Methods for the Detection of Hydroxychloroquine-Mediated Cardiotoxicity

(71) Applicant: Mayo Foundation for Medical Education and Research, Rochester, MN (US)

(72) Inventors: Geoffrey B. Johnson, Rochester, MN (US); Ian C. Chang, Rochester, MN (US); Martha Grogan, Rochester, MN (US); John P. Bois, Rochester, MN (US); Melanie C. Bois, Rochester, MN (US); Joseph J. Maleszewski, Rochester, MN (US)

(21) Appl. No.: 16/533,114

(22) Filed: Aug. 6, 2019

Related U.S. Application Data
(60) Provisional application No. 62/715,606, filed on Aug. 7, 2018.

(51) Int. Cl. A61K 51/02; A61B 6/03

(52) U.S. Cl. CPC A61K 51/025; A61B 6/032; A61B 6/037

(57) ABSTRACT

A method for detecting hydroxychloroquine-mediated cardiotoxicity in a subject. The method can comprise administering a radiotracer to the subject and acquiring an image to detect the presence or absence of hydroxychloroquine-mediated cardiotoxicity in the subject. If cardiotoxicity is found to be present, the method can comprise a further step of determining an extent of the cardiotoxicity in the subject.

### CROSS-REFERENCE TO RELATED APPLICATIONS

This application claims priority to U.S. Provisional Patent Application No. 62/715,606, filed on Aug. 7, 2018, the entirety of which is incorporated herein by reference.

### STATEMENT REGARDING FEDERALLY SPONSORED RESEARCH

Not applicable.

### BACKGROUND OF THE INVENTION

Hydroxychloroquine (HCQ) is a drug often used to treat rheumatologic diseases and prevent malaria infections. The drug has an unfortunate side effect. The drug can have toxic effects on the heart muscle, causing the heart to get thicker, and weaker. This side effect is called hydroxychloroquine-mediated cardiotoxicity. With current methods, the diagnosis of hydroxychloroquine-mediated cardiotoxicity is difficult to identify or confirm and requires an invasive biopsy, called an endomyocardial biopsy. When this biopsy is performed, a catheter is inserted into a large vein in the neck or the groin, and pushed into the heart chamber, where several small pieces of the heart muscle are removed. The diagnosis is made with pathologic studies of the biopsy tissue under a microscope. This procedure is invasive, carries risks, and is expensive. If given an alternative, most patients would prefer not to undergo a biopsy.

In light of the above, there is a dire need for an improved technology that provides a diagnosis of hydroxychloroquine-mediated cardiotoxicity without the inherent risks associated with existing techniques such as endomyocardial biopsies.

### SUMMARY OF THE INVENTION

The present disclosure overcomes the aforementioned shortcomings by providing systems and methods for detection of hydroxychloroquine-mediated cardiotoxicity using nuclear imaging. The present disclosure is based on the experimental discovery that HCQ-mediated cardiotoxicity can be targeted using a radiotracer. The present disclosure has potential to provide systems and methods that greatly reduce the expense and risks associated with traditional diagnoses.

In one aspect, the present disclosure provides a method for detecting hydroxychloroquine-mediated cardiotoxicity in a subject. The method can comprise administering a radiotracer to the subject and acquiring an image to detect the presence or absence of hydroxychloroquine-mediated cardiotoxicity in the subject.

The method may further comprise determining an extent of the cardiotoxicity in the subject if cardiotoxicity is present. The extent of the cardiotoxicity may include an estimate of the amount of hydroxychloroquine in the heart of the subject. Further, the radiotracer may be comprised of a gamma-emitting radioisotope. The radioisotope may be selected from the group consisting of ^{99m}Tc, ¹²³I, ¹¹¹In, ⁶⁷Ga, ¹⁷⁷Lu, ²⁰¹Ti, ^{117m}Sn, and ¹²⁵I. Additionally or alternatively, the radiotracer may comprise comprises pyrophosphate (PYP), 3,3-diphosphono-1,2-propanodicarboxylic acid (DPD), hydroxymethylene diphosphonate (HDP), or methylene diphosphonate (MDP). Additionally, the radiotracer can be ^{99m}Tc-pyrophosphate.

The acquired image can be acquired using planar scintigraphy or single-photon emission computed tomography. Additionally or alternatively, the image can be acquired using single-photon emission computed tomography with computed tomography. Additionally or alternatively, the image may be acquired using positron emission tomography.

In another aspect, the present disclosure provides a system for detecting hydroxychloroquine-mediated cardiotoxicity in a subject. The system can comprise an injector configured to administer a radiotracer to the subject and an imaging system configured to acquire an image, wherein the image provides information about the presence or absence of hydroxychloroquine-mediated cardiotoxicity in the subject.

The image may provide information as to the extent of the cardiotoxicity in the subject if a cardiotoxicity is present. The information as to the extent of the cardiotoxicity can include an estimate of the amount of hydroxychloroquine in the heart of the subject. Further, the radiotracer may comprise a gamma-emitting radioisotope. The radiotracer may comprise a radioisotope selected from the group consisting of ^{99m}Tc, ¹²³I, ¹¹¹In, ⁶⁷Ga, ¹⁷⁷Lu, ²⁰¹Ti, ^{117m}Sn, and ¹²⁵I. The radiotracer may comprise pyrophosphate (PYP), 3,3-diphosphono-1,2-propanodicarboxylic acid (DPD), hydroxymethylene diphosphonate (HDP), or methylene diphosphonate (MDP). Additionally, the radiotracer may be ^{99m}Tc-pyrophosphate.

The imaging system may be a planar scintigraphy system or a single-photon emission computed tomography system. Additionally or alternatively, the imaging system may be a single-photon emission computed tomography system with computed tomography. Additionally or alternatively, the imaging system may be a positron emission tomography system.

Various other features of the present invention will be made apparent from the following detailed description and the drawings.

### BRIEF DESCRIPTION OF THE DRAWINGS

FIG. 1 is a process flowchart for a method of detecting hydroxychloroquine-mediated cardiotoxicity in a subject.

FIG. 2 is a perspective view of an example of a single-photon emission computed tomography (SPECT) system that can be used with the systems and methods of the present disclosure.

FIG. 3 shows experimental imaging results of a subject with hydroxychloroquine-mediated cardiotoxicity, as presented in the Example below.

FIG. 4 shows experimental endomyocardial biopsy results of the subject with hydroxychloroquine-mediated cardiotoxicity, as presented in the Example below.

### DETAILED DESCRIPTION

As will be described, the present disclosure provides systems and methods for detection of hydroxychloroquine-mediated cardiotoxicity using nuclear imaging.

As used herein, “hydroxychloroquine” or “HCQ” carries its usual meaning and composition. Either of these terms may refer to hydroxychloroquine as a separate compound, or as hydroxychloroquine sulfate, which is often referred to as the brand name “Plaquenil®”.

As used herein, “cardiac region” refers to a region of the subject that includes the heart. In some cases, the cardiac region may also include other components of the chest of the subject such as the rib cage of the subject.

As used herein, “radiotracer uptake” may refer to the quantification of the amount of radioactive tracer present within a specific anatomical region or component.

As used herein, a “subject” may refer to a mammal. The subject may be a human. The human may be known to have been exposed to or treated with hydroxychloroquine in the past.

As used herein, “bone scintigraphy” may refer to PYP, DPD, MDP, or HDP planar and singe-photon emission computerized tomography (SPECT) imaging, or another similar method.

Although the systems and methods of the present disclosure are described as being primarily related to cardiotoxicity, one of skill in the art would recognize that the teachings of the present disclosure could readily by applied to other illnesses involving hydroxychloroquine or similar compounds such as chloroquine.

The new diagnostic method and system provided herein can be useful for a variety of assessment of treatment decisions relating HCQ-mediated cardiotoxicity. The teachings described herein can be used to confirm or reject the diagnosis of hydroxychloroquine-mediated cardiotoxicity as a cause of heart failure in a subject. Further, the method could be employed to direct clinical decisions to continue, change, or stop the use of hydroxychloroquine for a given subject. Additionally, the method can help a clinician develop a prognosis for how bad the patient's disease course will be in the future and therefore how aggressive a course of intervention is appropriate. Further yet, the diagnostic method could be used in follow-ups to see if the disease is progressing, stable, or resolving with therapy.

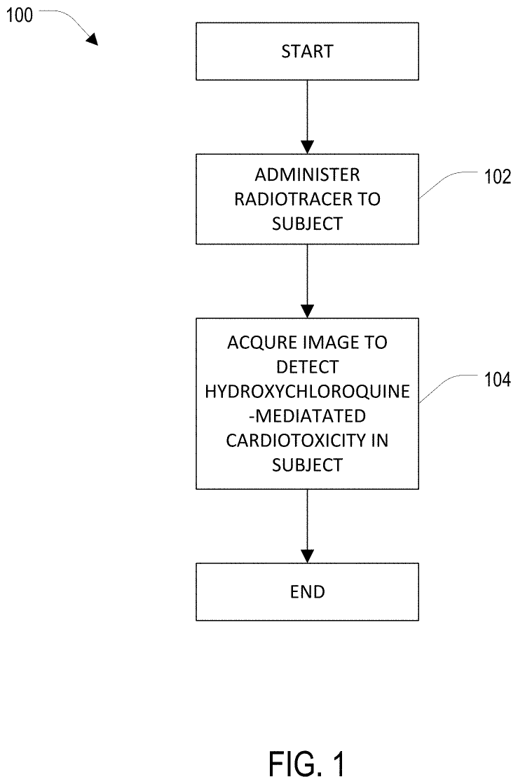

FIG. 1 depicts a process flowchart 100 for a method for detecting hydroxychloroquine-mediated cardiotoxicity in a subject. The method can comprise a first step of administering a radiotracer to the subject. The radiotracer may be administered by any article or system commonly known in the art for introducing radiotracers into a subject. For example, the radiotracer may be injected into the bloodstream of the subject using a radiotracer injector that has a needle.

The administered radiotracer may have a radioisotope. This radioisotope of the radiotracer may be selected from the group consisting of ^{99m}Tc, ¹²³I, ¹¹¹In, ⁶⁷Ga, ¹⁷⁷Lu, ²⁰¹Ti, ^{117m}Sn, ¹²⁵I, or any other commonly used gamma ray emitter. The radiotracer may specifically be ^{99m}Tc. The radiotracer may alternatively be a positron emitter such as ¹⁸F, ⁶⁸Ga, ¹¹C, ¹³N, ⁸²Rb, ¹⁵O or another commonly used positron emitter. The administered radiotracer may alternatively be a non-nuclear imaging agent such as an MRI based agent.

The radiotracer may comprise a group known to bind to calcium deposits. For example, the radiotracer may comprise pyrophosphate (PYP), 3,3-diphosphono-1,2-propanodicarboxylic acid (DPD), hydroxymethylene diphosphonate (HDP), or methylene diphosphonate (MDP), or another comparable compound. The radiotracer may be ^{99m}Tc-pyrophosphate.

The method 100 may comprise a second step of acquiring an image to detect the presence or absence of hydroxychloroquine-mediated cardiotoxicity in the subject. The image may be acquired from an imaging system. The image may be acquired using planar scintigraphy, SPECT, or a similar imaging process. The image may include a combination of nuclear imaging and anatomical imaging. For example, the image may be acquired using SPECT with computed tomography. The image may also be acquired using positron emission tomography or a similar process. The image may be of the cardiac region of the subject.

The method may further comprise determining an extent of the cardiotoxicity in the subject if a cardiotoxicity is found to be present. The extent of the cardiotoxicity may include an estimate of the amount of hydroxychloroquine in the heart of the subject. The extent of cardiotoxicity may be evaluated in different regions of the heart. The extent of the HCQ-mediated cardiotoxicity might be determined using quantitative scoring methods such as those previously reported for the diagnosis of other infiltrative cardiac diseases, such as transthyretin cardiac amyloidosis. These methods might include calculating a score from the radiotracer uptake of the heart either 2-dimensionally on the planar image or 3-dimensionally on the tomographic images after adjusting for background uptake. The background radiotracer uptake may be determined using a control region of the subject, such as the contralateral ribs of the subject or the blood pool in the heart. Such a radiotracer activity score of the heart could be serially followed to determine the change in cardiotoxicity over time or with treatment.

In another aspect, the present disclosure provides a system for detecting hydroxychloroquine-mediated cardiotoxicity in a subject. The system can comprise an injector configured to administer a radiotracer to the subject and an imaging system configured to acquire an image, wherein the image provides information about the presence or absence of hydroxychloroquine-mediated cardiotoxicity in the subject.

The radiotracer may have the same properties and compositions as described above in the method of FIG. 1. The injector may comprise a syringe with a needle.

The image may provide information as to the extent of the HCQ-mediated cardiotoxicity in the subject if any cardiotoxicity is found to be present. The information as to the extent of the cardiotoxicity may include or allow for a determination of the amount of hydroxychloroquine in the heart of the subject. The extent of cardiotoxicity may be determined for different regions of the heart. The image may provide a quantitative score corresponding to the cardiotoxicity in the heart of the subject.

The imaging system used to acquire the image may be a planar scintigraphy system or a SPECT system. The imaging system may be positron emission tomography (PET) system. The nuclear imaging system may be combined with another imaging system such as computed tomography (CT) or magnetic resonance imaging (MM).

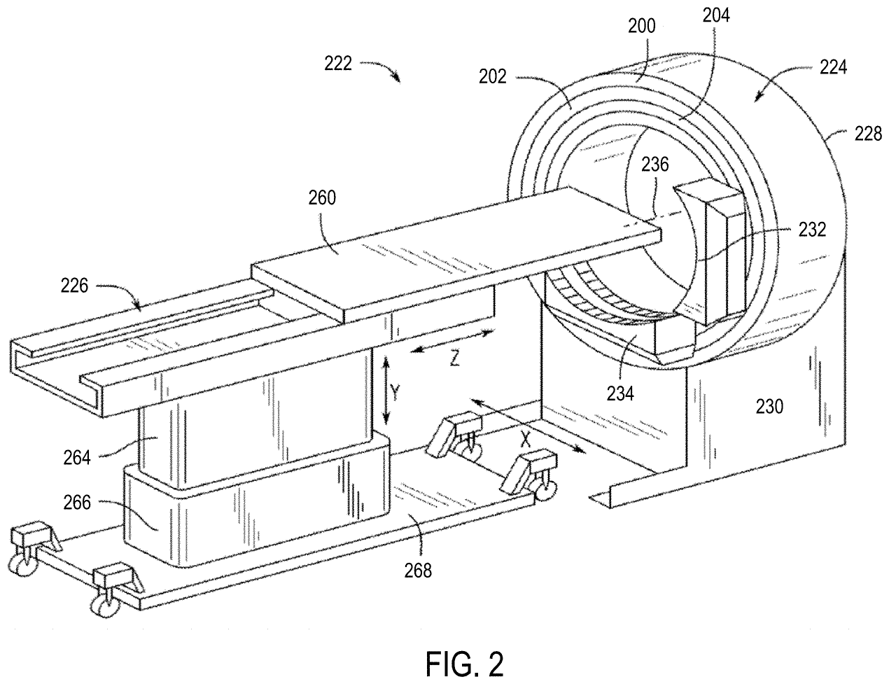

Referring particularly to FIG. 2, the imaging system of the method and system described herein may include a SPECT imaging system 222 that is illustrated and includes a tomography machine 224 and a patient support table 226. The table 226 includes a top surface 260 which allows supported movement of the top surface 260 along a scanning or horizontal Z-axis. The top surface 260 is supported by a vertical leg 264 which extends upwardly from a collar 266. The length of the leg 264 can be increased or decreased to raise or lower top surface 260 along a vertical Y-axis. The collar 266 is secured to a dolly 268 having four wheels. Thus, the table 226 enables an operator to position a subject on the top surface 260 in the bore of the tomographic machine 224.

The tomography machine 224 includes a pedestal 230, a gantry 228 and two annular detectors 232, 234. The top surface of the pedestal 230 receives an outer surface of gantry 228 and it houses a motor for rotating moving components of the gantry 228 about a central gantry rotation axis 236 as described in more detail below. The gantry 228 includes an annular race housing 200, which encircles first and second moveable rings 202, 204. Each of the rings 202 and 204 is annular shaped and when the machine 224 is assembled, all of the rings are concentric about imaging axis 236.

The detectors 232 and 234, depending upon configuration, may be stationary, while an annular collimator rotates to acquire different view angles. Alternatively, the detectors 232 and 234 and an associated collimator may be attached to one of the movable rings 202 and 204. Regardless of whether the detectors 232 and 234 are stationary or movable with the rings 202 and 204, the rings 202 and 204 may be unlocked from each other and rotated on their separate rings 202 and 204 to a number of different configurations. For example, they may be oriented 180 degrees apart for one scan and they may be oriented 90 degrees apart for another scan. The rings 202 and 204 are then locked together and rotated in unison during the scan to achieve the prescribed range of view angles.

As described herein, each camera 232, 234 has a collimator associated therewith. A scintillation crystal is positioned to absorb gamma emissions and produce light emissions corresponding to each absorbed gamma emission. The light emissions are directed toward an array of closely packed photomultiplier tubes (PMTs). Detected light emissions cause the PMTs to produce analog signals which are sent to a computer system that uses the signals to compute M and N coordinates of each gamma emission absorbed in terms of analog signal magnitudes. Alternatively, silicon photomultipliers (SPMs) or “digital” detectors may be used in the SPECT system.

### EXAMPLE

The following example is provided in order to demonstrate and further illustrate certain embodiments and aspects of the present disclosure and is not to be construed as limiting the scope of the disclosure.

Imaging using 99mTc-PYP has recently been shown to have high specificity for a different cause of heart failure: transthyretin cardiac amyloidosis (ATTR). In fact, in patients without evidence of plasma cell dyscrasia (that causes a different heart disease, light chain cardiac amyloidosis), the specificity is near 100%. Therefore, there has been a significant increase in the clinical volume of ^{99m}Tc-PYP scans in patients with heart failure to assess for ATTR cardiac amyloidosis. We made the discovery of the ability to detect hydroxychloroquine-mediated cardiotoxicity with a ^{99m}Tc-PYP scan serendipitously. HCQ-mediated cardiotoxicity was not previously thought to be a disease involving the laying down of calcium (which PYP is known to bind), nor is it caused by a type of amyloidosis like ATTR (which PYP is known to bind). HCQ-mediated cardiotoxicity is instead a disease caused by the drug accumulating in the heart muscle. Consequently, one of skill in the art would not have expected such a finding. We have now seen several cases confirming this finding.

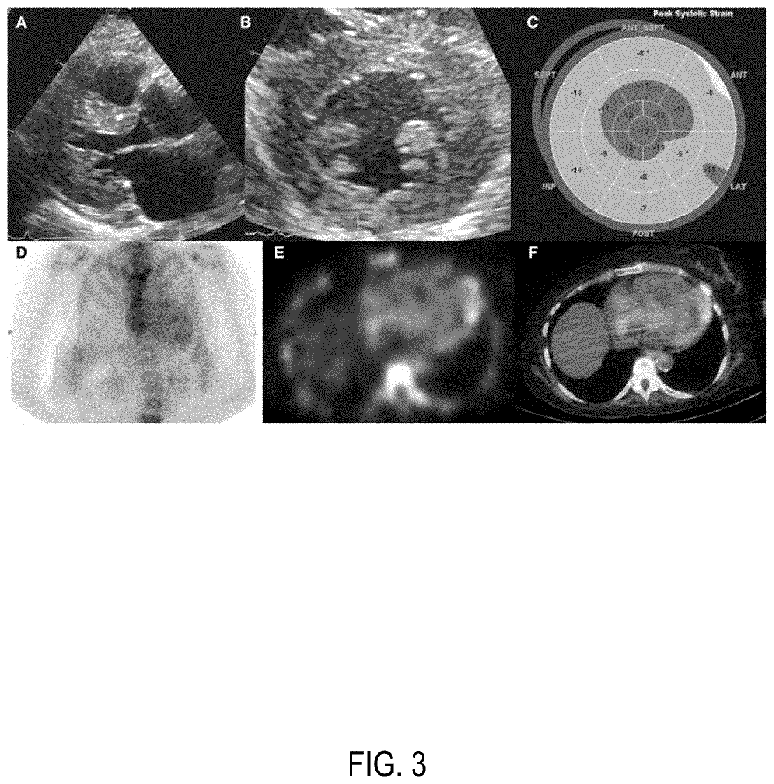

The experimental study included a 69-year-old woman with Sjogren syndrome on hydroxychloroquine for 15 years presented with congestive heart failure. On presentation, the patient's creatinine and estimated glomerular filtration rate were 1.2 mg/dL and 45 mL/min per m², respectively. Transthoracic echocardiogram (A of FIG. 3: parasternal long axis; B of FIG. 3: parasternal short axis; C of FIG. 3: strain imaging) showed concentric left ventricular wall thickening and abnormal global averaged longitudinal peak systolic strain at −10%, most preserved at the apex. Planar scintigraphy (D of FIG. 3) of ^{99m}Tc-PYP uptake allows count comparison between the heart (right) with the contralateral chest (left) on 3-hour uptake anterior planar imaging. The high heart/contralateral ratio was 1.8 (>1.3 considered positive). Axial single photon emission tomography (E of FIG. 3) and fused single photon emission tomography/computed tomography imaging (F of FIG. 3) demonstrated myocardial uptake, most pronounced in the left ventricular lateral wall with a semiquantitative score of 2 (0=no cardiac uptake, 1=less than bone, 2=same as bone, and 3=greater than bone/rib) consistent with ATTR.

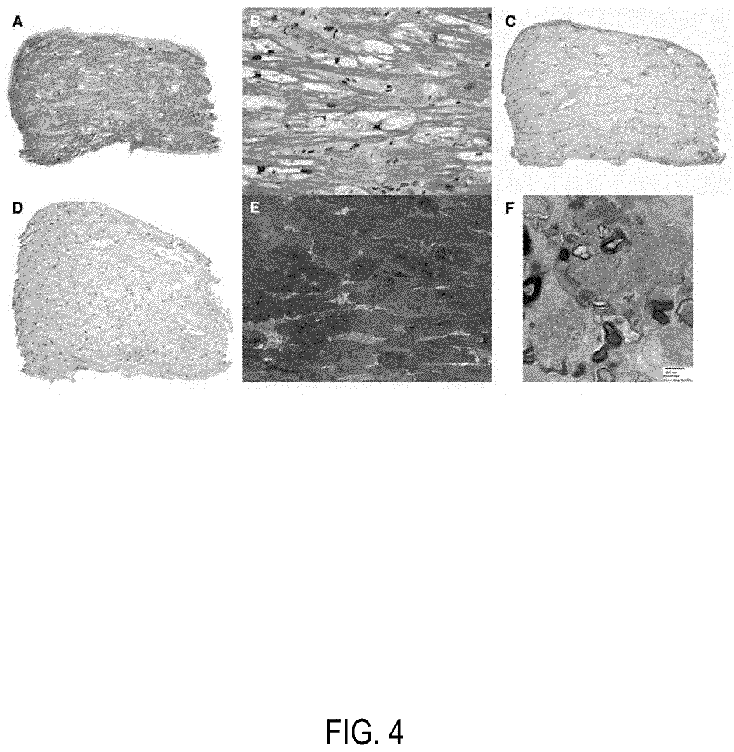

An endomyocardial biopsy was performed on the same subject. Hematoxylin and eosin-stained sections of the endomyocardial biopsy showed marked sarcoplasmic vacuolization of cardiac myocytes (A of FIG. 4 and B of FIGS. 4; ×100 and ×400 original magnification, respectively). Amyloid was not present on special stains, including sulfated Alcian blue stain and Congo red stains (C of FIG. 4 and D of FIG. 4, respectively, ×100 original magnification). Toluidine blue-stained thick sections show dense sarcoplasmic inclusions (E of FIG. 4; ×400 original magnification). Electron microscopy demonstrates the presence of myelinoid and curvilinear bodies, the combination of which is in keeping with hydroxychloroquine-mediated cardiotoxicity (F of FIG. 4), instead of a presumed diagnosis of cardiac ATTR from a falsely positive ^{99m}Tc-PYP scan.

Deemed positive using the conventional high heart/contralateral ratio and semi-quantitative scoring method, this case illustrates a previously unrecognized pathogenesis behind a positive cardiac uptake of bone scintigraphy ^{99m}Tc-PYP tracer in patients without ATTR or light chain amyloidosis. We envision that further studies will determine the mechanism of myocardial uptake of ^{99m}Tc-PYP as the use of this diagnostic modality increases.

With this experiment, we have identified that hydroxychloroquine-mediated cardiotoxicity can be detected with a 99m-technetium-labeled pyrophosphate (^{99m}Tc-PYP) planar and SPECT/CT scan, which was previously unrecognized. We identified positive binding of PYP in the heart muscle in a patient who later had an endomyocardial biopsy. The diagnosis of hydroxychloroquine-mediated cardiotoxicity was confirmed by light microscopy and transmission electron microscopy by a cardiovascular pathologist. Consequently, one could therefore use a ^{99m}Tc-PYP scan as a novel diagnostic imaging biomarker to detect the presence of hydroxychloroquine deposition in the myocardium in patients with heart failure. With the specific imaging techniques and analysis methods we have developed, we envision it being further possible to also estimate the burden of hydroxychloroquine in the heart muscle.

Thus, the present invention provides systems and methods for enhanced diagnosis of transthyretin-related cardiac amyloidosis in a subject.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

We claim:

1. A method for detecting hydroxychloroquine-mediated cardiotoxicity in a subject, the method comprising:
administering a radiotracer to the subject;
acquiring an image to detect the presence or absence of hydroxychloroquine-mediated cardiotoxicity in the subject.

2. The method of claim 1, further comprising:
determining an extent of the cardiotoxicity in the subject if cardiotoxicity is present.

3. The method of claim 2, wherein the extent of the cardiotoxicity includes an estimate of the amount of hydroxychloroquine in the heart of the subject.

4. The method of claim 1, wherein the radiotracer comprises a gamma-emitting radioisotope.

5. The method of claim 4, wherein the radiotracer comprises a radioisotope selected from the group consisting of ^{99m}Tc, ¹²³I, ¹¹¹In, ⁶⁷Ga, ¹⁷⁷Lu, ²⁰¹Ti, ^{117m}Sn, and ¹²⁵I.

6. The method of claim 1, wherein the radiotracer comprises pyrophosphate (PYP), 3,3-diphosphono-1,2-propanodicarboxylic acid (DPD), hydroxymethylene diphosphonate (HDP), or methylene diphosphonate (MDP).

7. The method of claim 6, wherein the radiotracer is ^{99m}Tc-pyrophosphate.

8. The method of claim 1, wherein the image is acquired using planar scintigraphy or single-photon emission computed tomography.

9. The method of claim 8, wherein the image is acquired using single-photon emission computed tomography with computed tomography.

10. The method of claim 1, wherein the image is acquired using positron emission tomography.

11. A system for detecting hydroxychloroquine-mediated cardiotoxicity in a subject, the system comprising:
an injector configured to administer a radiotracer to the subject;
an imaging system configured acquire an image, wherein the image provides information about the presence or absence of hydroxychloroquine-mediated cardiotoxicity in the subject.

12. The system of claim 11, wherein the image provides information as to the extent of the cardiotoxicity in the subject if a cardiotoxicity is present.

13. The system of claim 12, wherein the information as to the extent of the cardiotoxicity includes an estimate of the amount of hydroxychloroquine in the heart of the subject.

14. The system of claim 11, wherein the radiotracer comprises a gamma-emitting radioisotope.

15. The system of claim 14, wherein the radiotracer comprises a radioisotope selected from the group consisting of ^{99m}Tc, ¹²³I, ¹¹¹In, ⁶⁷Ga, ¹⁷⁷Lu, ²⁰¹Ti, ^{117m}Sn, and ¹²⁵I.

16. The system of claim 11, wherein the radiotracer comprises pyrophosphate (PYP), 3,3-diphosphono-1,2-propanodicarboxylic acid (DPD), hydroxymethylene diphosphonate (HDP), or methylene diphosphonate (MDP).

17. The system of claim 16, wherein the radiotracer is ^{99m}Tc-pyrophosphate.

18. The system of claim 11, wherein the imaging system is a planar scintigraphy system or single-photon emission computed tomography system.

19. The system of claim 18, wherein the imaging system is a single-photon emission computed tomography system with computed tomography.

20. The system of claim 11, wherein the imaging system is a positron emission tomography system.
